(19) United States
(12) Patent Application Publication
(10) Pub. No.: US 2018/0028631 A1
(43) Pub. Date: Feb. 1, 2018

(54) ANTI-SSEA4 CHIMERIC ANTIGEN RECEPTORS AND THEIR USE FOR TREATING CANCER

(71) Applicant: Lan Bo Chen, Lexington, MA (US)

(72) Inventor: Lan Bo Chen, Lexington, MA (US)

(21) Appl. No.: 15/664,098

(22) Filed: Jul. 31, 2017

Related U.S. Application Data
(60) Provisional application No. 62/368,637, filed on Jul. 29, 2016.

(51) Int. Cl. A61K 39/00; C07K 14/705; C07K 14/725; C07K 16/30; A61K 39/395

(52) U.S. Cl. CPC A61K 39/0011; C07K 16/30; A61K 39/39558; C07K 14/7051; C07K 14/70521; C07K 14/70578; C07K 2317/622; C07K 2317/64; C07K 2317/31; A61K 2039/5156; C07K 2319/74; C07K 2317/24

(57) ABSTRACT

A chimeric antigen receptor containing (i) a single chain Fv that specifically binds to stage-specific embryonic antigen 4 and (ii) an endodomain from CD3ζ or FcεRIγ. Also provided is a nucleic acid encoding the chimeric antigen receptor and an expression vector that contains the nucleic acid operably linked to a promoter that is active in T cells. Furthermore, two similar methods for treating a tumor are disclosed. The first method includes (i) obtaining T cells from a subject, (ii) transducing the T cells in vitro with an expression vector encoding a chimeric antigen receptor that contains an scFv specifically recognizing stage-specific embryonic antigen 4, (iii) expanding the transduced T cells in vitro, and (iv) infusing the expanded transduced T cells into the subject. The second method includes, in place of step (ii) above, transducing the T cells in vitro with an expression vector encoding a chimeric antigen receptor that is specific for a tumor antigen other than stage-specific embryonic antigen 4, and further requires a step of administering an antibody against stage-specific embryonic antigen 4.

### CROSS-REFERENCE TO RELATED APPLICATIONS

The present application claims priority to Provisional Application No. 62/368,637, filed on Jul. 29, 2016. The content of this prior application is hereby incorporated by reference in its entirety.

### BACKGROUND

Targeted cancer immunotherapy, as compared to chemotherapy, holds the promise of better efficacy, both short-term and long-term, with fewer side effects.

For example, anti-cancer vaccines utilizing a tumor antigen have been developed that stimulate a patient's own immune system to develop anti-tumor T cells. These T cells have a T cell receptor that can recognize the tumor antigen presented on tumor cell surfaces, which leads to T cell activation and eradication of the tumor cells.

Such an approach often loses effectiveness over time as a result of a T cell inhibitory environment in the tumor. The inhibitory environment blocks T cell activation, in part, by preventing co-stimulatory signals from working in conjunction with a primary signal initiated upon tumor antigen binding to the T cell receptor.

Recently, chimeric antigen receptors (“CARs”) have been developed to obviate the need for co-stimulatory signals upon antigen binding to the T cell receptor. A CAR contains (i) an extracellular domain that binds to the tumor antigen and (ii) one or more intracellular domains that provide both primary and co-stimulatory signals to the T cells. T cells can be engineered in vitro to express CAR having an extracellular domain of choice.

The CAR approach has proven to be effective, yet not without serious side effects. In an example, activation of a large number of T cells expressing CAR causes cytokine release syndrome. This syndrome, characterized by high fever, hypotension, and hypoxia, can result in multi-organ failure.

There is a need to develop CAR-based tumor therapies that are safer and more effective than those currently in use.

### SUMMARY

To meet this need, a chimeric antigen receptor (“CAR”) is provided. The CAR contains a single chain Fv (“scFv”) that specifically binds to stage-specific embryonic antigen 4 (“SSEA4”) and an endodomain from CD3ζ or FcεRIγ.

Also provided is a nucleic acid encoding the just-described CAR and an expression vector that contains the nucleic acid operably linked to a promoter that is active in T cells.

Furthermore, a method for treating a tumor in a subject is disclosed. The method includes (i) obtaining T cells from the subject, (ii) transducing the T cells in vitro with an expression vector encoding a CAR that contains an scFv specifically recognizing SSEA4, (iii) expanding the transduced T cells in vitro, and (iv) infusing the expanded transduced T cells into the subject. The method results in raising an anti-tumor T cell response.

Another method of the invention includes the steps of (i) obtaining T cells from a subject, (ii) transducing the T cells in vitro with an expression vector encoding a CAR, (iii) expanding the transduced T cells in vitro, (iv) infusing the expanded transduced T cells into the subject having a tumor, and (v) administering an antibody that specifically binds to SSEA4.

The details of one or more embodiments of the invention are set forth in the description below. Other features, objects, and advantages of the invention will be apparent from the description and from the claims.

Importantly, all documents cited herein are hereby incorporated by reference in their entirety.

### DETAILED DESCRIPTION

As mentioned above, a CAR of the invention contains an scFv that specifically binds to SSEA4. Examples of an scFv that specifically binds to SSEA4 are described in US Patent Application Publication 2016/0102151.

In addition to the scFv, the CAR also contains an endodomain from CD3ζ or FcεRIγ. The endodomain contains one or more immunoreceptor tyrosine-based activating motifs (“ITAM”).

The CAR includes a hinge/spacer region and a transmembrane region between the scFv and the endodomain.

Exemplary sequences that can be used as a hinge/spacer region are derived from the hinge region of, e.g., IgG1, IgG4, and IgD. Alternatively, it can be derived from CD8. See, e.g., Dai et al. 2016, J. Natl. Cancer Inst. 108:1-14 (“Dai et al.”) and Shirasu et al., 2012, Anticancer Res. 32:2377-2384 (“Shirasu et al.”).

Exemplary transmembrane regions that can be included in the CAR are derived from CD3ζ, CD4, CD8, or CD28. See Dai et al. and Shirasu et al.

Optionally, the CAR also contains a second endodomain in addition to the endodomain from CD3ζ or FcεRIγ. The second endodomain, e,g., from CD28, CD137, CD4, OX40, or ICOS, contains one or more ITAM.

In the CAR having a second endodomain, the scFv is fused via the hinge/spacer region to the second endodomain and the second endodomain is fused to the first endodomain from CD3ζ or FcεRIγ.

Another CAR of the invention contains a third endodomain, which also can be from CD28, CD137, CD4, OX40, or ICOS. The third endodomain is different from the second endodomain. This CAR is arranged such that the scFv is fused via the hinge/spacer region/transmembrane domain to the third endodomain, the third endodomain is fused to the second endodomain, and the second endodomain is fused to the first endodomain from CD3ζ or FcεRIγ.

In a specific embodiment, the CAR contains an anti-SSEA4 scFv fused to a spacer/hinge that is fused to a transmembrane domain fused to the N-terminus of the endodomain from CD28, which in turn is fused to the N-terminus of the endodomain from CD137, which in turn is fused to the N-terminus of the endodomain from CD3ζ.

Nucleic acids encoding any of the CAR described, supra, are also within the scope of the invention. The nucleic acids of the invention can be constructed by recombinant technology known in the art.

Further within the scope of the invention is an expression vector including the CAR-encoding nucleic acid operably linked to a promoter. The promoter is active in T cells.

Exemplary CAR expression vectors based on lentiviral vectors or a gamma retroviral vectors are set forth in Dai et al.; Jin et al. 2016, EMBO Mol. Med. 8:702-711; Liechtenstein et al. 2013, Cancers 5:815-837; and Schonfeld et al. 2015, Mol. Therapy 23:330-338. Such expression vectors are used for integrating the promoter/CAR-encoding nucleic acid into T cell genomic DNA to produce stable expression of the CAR.

In another embodiment, the expression vector contains sequences that facilitate transposon-mediated genomic integration into T cells of the promoter/CAR-encoding nucleic acid. Examples of these expression vectors are the so-called “PiggyBac” and “Sleeping Beauty” expression vectors. See Nakazawa et al. 2011, Mol. Ther. 19:2133-2143 and Sourindra et al. 2013, J. Immunotherapy 36:112-123.

Moreover, the invention encompasses a first method of for treating a tumor with the CAR set forth above. The method is effective for treating, e.g., a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

The first method includes obtaining T cells from a subject suffering from a tumor. Procedures for isolating T cells are known in the art. See, e.g., Kaiser et al. 2015, Cancer Gene Therapy 22:72-78 (“Kaiser et al.”). In a particular embodiment, CD8⁺ cells are obtained from the subject.

The T cells are transduced in vitro with a CAR containing a scFv that specifically recognizes SSEA4, i.e., any of the CAR described above. Transduction of T cells is performed by electroporation, lipofection, lentiviral infection, or gamma retrovirus infection.

The transduced T cells are expanded in vitro, using methods known in the art. See Kaiser et al.

Finally, the expanded T cells are infused in one batch or in two or more batches into the subject having a tumor.

In one embodiment, the first method of the invention includes a preconditioning step that is performed prior to the just-mentioned infusion step. The preconditioning step is accomplished by treating the subject with a drug that induces lymphodepletion. Examples of these drugs include cyclophosphamide and fludarabine. Additional drug examples can be found in Dai et al. and Han et al. 2013, J. Hematol. Oncol. 6:47-53.

The first method for treating a tumor, set forth in the preceding paragraphs, can also include administering an antibody or antibody fragment that specifically binds to SSEA4. Examples of an antibody that specifically binds to SSEA4 include a chimeric anti-SSEA4 antibody and a fully humanized anti-SSEA4 monoclonal antibody. An antibody fragment that specifically binds to SSEA4 can be, but is not limited to, an anti-SSEA4 Fab and an anti-SSEA4 scFv. See US Patent Application Publication 2016/0102151 for more examples of anti-SSEA4 antibodies and anti-SSEA4 antibody fragments for use in the method of the invention are described in.

The anti-SSEA4 antibody or antibody fragment can be linked to a cytokine, a cytotoxic agent, a modified immunoglobulin Fc domain, anti-CD3, or anti-CD16.

A cytokine can be fused to the anti-SSEA4 antibody or antibody fragment as part of a fusion protein. See Kiefer et al. 2016, Immunol. Revs. 270:178-192. In another example, the cytokine is linked to the anti-SSEA4 antibody or antibody fragment via cross-links between lysine residues. Exemplary suitable cytokines include G-CSF, GM-CSF, IFNγ, IFNα, IL-1β, IL-2, IL-4, IL-6, IL-7, IL-9, IL-12, IL-13, IL-15, IL-17, IL-21, IL-23, and TNF.

Exemplary cytotoxic agents are diphtheria toxin, pseudomonas exotoxin A (“PE38”), doxorubicin, methotrexate, an auristatin, a maytansine, a calicheamicin, a duocarmycin, a pyrrolobenzodiazepine dimer, and 7-ethyl-10-hydroxy-camptothecin. Suitable cytotoxic agents are described in Peters et al. 2015, Biosci. Rep. 35:1-20 (“Peters et al”); Bouchard et al. 2014, Bioorg. Med. Chem. Lett. 24:5357-5363; Panowski et al. 2014, mAbs 6:34-45; and Mazor et al. 2016, Immunol. Revs. 270:152-164.

The cytotoxic agent can be linked to the anti-SSEA4 antibody or antibody fragment via a linker. In an embodiment, the linker is cleavable such that, upon internalization of the anti-SSEA4 antibody or antibody fragment by a tumor cell, the cytotoxic agent is cleaved from the anti-SSEA4 antibody or antibody fragment. Examples of a cleavable linker include, but are not limited to, acid-labile small organic molecules (e.g., hydrazone), protease cleavable peptides (e.g., valine-citrulline dipeptide), and disulfide bonds. In another embodiment, the linker is not cleavable. In this case, the cytotoxic agent is released upon degradation of the anti-SSEA4 antibody or antibody fragment linked to it. Additional examples of linkers are described in Peters et al.

If the cytotoxic agent is a protein, it can be linked to the anti-SSEA4 antibody or antibody fragment via a peptide bond, e.g., as part of a fusion protein. In a particular example, PE38 can be fused to the C-terminus of a V_{L} chain of an anti-SSEA4 monoclonal antibody.

In a particular embodiment, the first method for treating a tumor is carried out by administering an anti-SSEA4 antibody fragment linked to a modified immunoglobulin Fc domain together with the CAR-expressing T cells. For example, the Fc domain can be modified such that it targets the FcγRIIa receptor, the FcγRIIIa receptor, or the FcRn receptor, as compared to an unmodified Fc domain. Targeting the FcγRIIa or FcγRIIIa receptor leads to an increased cytotoxic immune response. On the other hand, targeting the FcRn receptor increases the half-life of the anti-SSEA4 antibody fragment. Modifications to the Fc domain that increase its affinity for the FcγRIIa receptor, the FcγRIIIa receptor, or the FcRn receptor are described in Moore et al. 2010, mAbs 2:181-189 and Lobner et al. 2016, Immunol. Revs. 270:113-131.

In another embodiment, the anti-SSEA4 antibody fragment is linked to an anti-CD3 molecule. The anti-CD3 molecule activates T cells localized to tumor cells via the anti-SSEA4 antibody fragment. An exemplary anti-CD3 molecule is an antibody fragment. The anti-CD3 molecule can specifically bind to CD3ε. Further, an scFv that specifically binds to SSEA4 can be fused to another scFv that specifically binds to CD3.

In still another embodiment, the anti-SSEA4 antibody fragment is linked to an anti-CD16 molecule. The anti-CD16 molecule activates NK cells localized to tumor cells via the anti-SSEA4 antibody fragment. Like the anti-CD3 molecule described in the preceding paragraph, the anti-CD16 molecule can be an antibody fragment that binds specifically to CD16. Exemplary constructs are an anti-SSEA4/anti-CD16 chimeric antibody and a scFv that specifically binds to SSEA4 fused to another scFv that specifically binds to CD16.

A second method for treating a tumor is also provided. The second method, like the first method, can also be used for treating a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

The second method requires the steps of obtaining T cells from a subject having a tumor, transducing the T cells in vitro with an expression vector that encodes a CAR, expanding the transduced T cells in vitro, infusing the expanded transduced T cells into the subject, and administering an antibody that specifically binds to SSEA4.

This method employs an expression vector that encodes a CAR having a different target than the target used in the first method, i.e., SSEA4. The CAR utilized in the second method specifically binds to the following targets: α-folate receptor, CD19, CD20, CAIX, CD22, CD30, CD33, CD44v7/8, CEA, EGP-2, EGP-40, erb-B2, erb-B3, erb-B4, FBP, fetal acetylcholine receptor, GD2, GD3, Her2/neu, IL-13R-α2, KDR, kappa light chain, LeY, L1, MAGE-A1, mesothelin, MUC1, NKG2D ligand, h5T4, PSCA, PSMA, TAG-72, or VEGF-R2. The specific CAR is selected depending on the presence of the target in the tumor to be treated. For example, a CAR that specifically binds to CD19 can be used for treating a B-cell tumor.

The second method uses the same procedures for obtaining, transducing, expanding, and infusing the T cells into the subject as the first method Like the first method, it also includes administering an antibody that specifically binds to SSEA4. The antibody can be linked to a cytokine, a cytotoxic agent, a modified immunoglobulin Fc domain, anti-CD3, and anti-CD16 as set forth above.

Without further elaboration, it is believed that one skilled in the art can, based on the description above, utilize the present invention to its fullest extent.

The following references, some cited supra, can be used to better understand the background of the application:

Abate-Daga et al., Mol. Ther. Oncolytics 3:1-7.

Bouchard et al. 2014, Bioorg. Med. Chem. Lett. 24:5357-5363

Curran et al. 2012, J. Gene Med. 14:405-415

Dai et al. 2016, J. Natl. Cancer Inst. 108:1-14

Guest et al., 2005, J. Immunother. 28:203-211

Han et al. 2013, J. Hematol. Oncol. 6:47-53

James et al. 2008, J. Immunol. 180:7028-7038.

Kaiser et al. 2015, Cancer Gene Therapy 22:72-78.

Kiefer et al. 2016, Immunol. Revs. 270:178-192

Lobner et al. 2016, Immunol. Revs. 270:113-131

Mazor et al. 2016, Immunol. Revs. 270:152-164

Moore et al. 2010, mAbs 2:181-189

Moritz et al. 1995 Gene Therapy 2:539-546

Nakazawa et al. 2011, Mol. Ther. 19:2133-2143

Panowski et al. 2014, mAbs 6:34-45

Peters et al. 2015, Biosci. Rep. 35:1-20

Rodgers et al. 2016, Proc. Natl. Acad. Sci. Jan. 12:E459-E468

Schonfeld et al. 2015, Mol. Therapy 23:330-338

Shirasu et al. 2012, Anticancer Res. 32:2377-2384

Sourindra et al., 2013, J. Immunotherapy 36:112-123

The contents of the above references are hereby incorporated by reference in their entirety.

### OTHER EMBODIMENTS

All of the features disclosed in this specification may be combined in any combination. Each feature disclosed in this specification may be replaced by an alternative feature serving the same, equivalent, or similar purpose. Unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A chimeric antigen receptor, comprising a single chain Fv (scFv) that specifically binds to stage-specific embryonic antigen 4 (SSEA4), and a first endodomain from CD3ζ or FcεRIγ.

2. The chimeric antigen receptor of claim 1, further comprising a second endodomain from CD28, CD137, CD4, OX40, or ICOS, wherein the scFv is fused to the second endodomain and the second endodomain is fused to the first endodomain.

3. A nucleic acid encoding the chimeric antigen receptor of claim 2.

4. An expression vector comprising the nucleic acid of claim 3 operably linked to a promoter, wherein the promoter is active in T cells.

5. The chimeric antigen receptor of claim 2, further comprising a third endodomain from CD28, CD137, CD4, OX40, or ICOS, wherein the third endodomain is different from the second endodomain and the scFv is fused to the second endodomain via the third endodomain.

6. A nucleic acid encoding the chimeric antigen receptor of claim 5.

7. An expression vector comprising the nucleic acid of claim 6 operably linked to a promoter, wherein the promoter is active in T cells.

8. The chimeric antigen receptor of claim 5, wherein the scFv is fused to the N-terminus of the second endodomain from CD28, the second endodomain is fused to the N-terminus of the third endodomain from CD137, and the third endodomain is fused to the N-terminus of the first endodomain from CD3ζ.

9. A nucleic acid encoding the chimeric antigen receptor of claim 8.

10. An expression vector comprising the nucleic acid of claim 9 operably linked to a promoter, wherein the promoter is active in T cells.

11. A method for treating a tumor in a subject, the method comprising:
obtaining T cells from the subject;
transducing the T cells in vitro with an expression vector encoding a chimeric antigen receptor (CAR) containing a scFv that specifically recognizes stage-specific embryonic antigen 4 (SSEA4), whereby the transduced T cells express the CAR;
expanding the transduced T cells in vitro; and
infusing the expanded transduced T cells into the subject having a tumor, whereby an anti-tumor T cell response is raised.

12. The method of claim 11, wherein the CAR contains a CD3ζ endodomain or an FcεRIγ endodomain.

13. The method of claim 12, wherein the CAR further contains an endodomain from CD28, CD137, CD4, OX40, or ICOS.

14. The method of claim 13, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

15. The method of claim 13, further comprising administering an antibody or antibody fragment that specifically binds to SSEA4.

16. The method of claim 15, wherein the antibody or antibody fragment is linked to a cytokine, a cytotoxic agent, a modified immunoglobulin Fc domain, anti-CD3, or anti-CD16.

17. The method of claim 16, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

18. The method of claim 17, wherein the antibody fragment is linked to a cytokine selected from the group consisting of G-CSF, GM-CSF, IFNγ, IFNα, IL-1β, IL-2, IL-4, IL-6, IL-7, IL-9, IL-12, IL-13, IL-15, IL-17, IL-21, IL-23, and TNF.

19. The method of claim 17, wherein the antibody fragment is linked to a cytotoxic agent selected from the group consisting of Diphtheria toxin, Pseudomonas exotoxin A, doxorubicin, methotrexate, an auristatin, a maytansine, a calicheamicin, a duocarmycin, a pyrrolobenzodiazepine dimer, and 7-ethyl-10-hydroxy-camptothecin.

20. The method of claim 17, wherein the antibody fragment is linked to a modified immunoglobulin Fc domain modified to target the FcγRIIa receptor, the FcγRIIIa receptor, or the FcRn receptor.

21. The method of claim 17, wherein the antibody fragment is linked to anti-CD3 or anti-CD16.

22. A method for treating a tumor in a subject, the method comprising:
obtaining T cells from the subject;
transducing the T cells in vitro with an expression vector that encodes a chimeric antigen receptor (CAR), whereby the transduced T cells express the CAR;
expanding the transduced T cells in vitro;
infusing the expanded transduced T cells into the subject having a tumor, whereby an anti-tumor T cell response is raised; and
administering an antibody that specifically binds to SSEA4.

23. The method of claim 22, wherein the CAR specifically binds to α-folate receptor, CD19, CD20, CAIX, CD22, CD30, CD33, CD44v7/8, CEA, EGP-2, EGP-40, erb-B2, erb-B3, erb-B4, FBP, fetal acetylcholine receptor, GD2, GD3, Her2/neu, IL-13R-α2, KDR, kappa light chain, LeY, L1, MAGE-A1, mesothelin, MUC1, NKG2D ligand, h5T4, PSCA, PSMA, TAG-72, or VEGF-R2.

24. The method of claim 23, wherein the antibody that specifically binds to SSEA4 is linked to an agent selected from the group consisting of a cytokine, a cytotoxic agent, a modified immunoglobulin Fc domain, anti-CD3, and anti-CD16.
